(19) United States
(12) Patent Application Publication
(10) Pub. No.: US 2003/0165403 A1
(43) Pub. Date: Sep. 4, 2003

(54) Decontamination of surface exposed to biological warfare agents

(76) Inventor: James L. Marsden, Manhattan, KS (US)

Correspondence Address: Mark Rogers, Speed & Rogers, 1701 Centerview, Suite 125, Little Rock, AR, 72211, US

(21) Appl. No.: 10/258,086

(22) PCT Filed: Mar. 28, 2001
(86) PCT No.: PCT/US01/09922

(51) Int. Cl. A61L 2/18; C02F 1/50

(52) U.S. Cl. 422/28; 210/764

(57) ABSTRACT

A system is disclosed in which a surface exposed to, suspected of being exposed to, or in imminent danger of being exposed to a biological warfare agent is treated with an effective amount of a quaternary ammonium compound to inactivate the biological warfare agent. The quaternary ammonium compound is preferably selected from the group consisting of an alkylpyridinium salt, a tetra-alkyl ammonium salt, and an alkylalicyclic ammonium salt. The quaternary ammonium salt is more preferably an alkylpyridinium salt and is most preferably cetylpyridinium chloride (“CPC”). The compound is preferably present in solution in a weight percent that is substantially within a range of from approximately 0.5% to approximately 5.0% and is more preferably present in solution in a weight percent of approximately 1%. The compound may be applied as a mist, spray, or fog. Air or other gases that have been exposed to or that are suspected of being exposed to a biological warfare agent may also be treated by passing the air or gas through a filter impregnated with an effective amount of a quaternary ammonium compound to inactivate the biological warfare agent.

### BACKGROUND OF THE INVENTION

This invention relates to a system for decontaminating surfaces exposed to biological warfare agents, and more particularly, to a system for treating such surfaces with a compound that inactivates a broad spectrum of biological warfare agents.

Current biological defense systems are based primarily on

1) prevention of attack through deterrence or preemptive destruction of biological weapons;

2) detection of biological agents in the event of an attack;

3) immunizations of military personnel;

4) protection of personnel using shelters or protective masks; and

5) treatment of infected personnel after an attack.

Each of these steps or activities serves an important function and offers advantages. Still, none of these steps accomplishes a thorough decontamination of environments, equipment, personnel, animals, or food in the event of an attack. Decontamination is an important part of reducing the impact of an attack using a biological warfare agent. For example, Bacillus anthracis is a sporeforming human and animal pathogen that causes anthrax. This organism is feared as a biological warfare agent and poses considerable difficulty in decontamination because of its sporeforming nature. Unless effective decontamination steps are taken, this organism can withstand high heat, UV light, chemical disinfectants, and drying conditions to persist in the environment for extended periods of time.

### SUMMARY OF THE INVENTION

It is therefore an object of the present invention to provide a system for decontaminating surfaces exposed to, suspected of being exposed to, or in imminent danger of being exposed to a biological warfare agent.

It is a further object of the present invention to provide for the systematic decontamination of environments, equipment, personnel, animals, and food exposed to, suspected of being exposed to, or in imminent danger of being exposed to a biological warfare agent.

It is a further object of the present invention to provide a system of the above type that uses a quaternary ammonium compound that inactivates a broad spectrum of biological warfare agents.

It is a further object of the present invention to provide a system of the above type that uses a quaternary ammonium compound that provides a residual anti-microbial effect.

It is a still further object of the present invention to provide a system of the above type in which cetylpyridinium chloride is used to treat surfaces exposed to, suspected of being exposed to, or in imminent danger of being exposed to a biological warfare agent.

It is a still further object of the present invention to provide a system of the above type in which a compound may be atomized, fogged, misted, or sprayed from portable or stationary equipment, vehicles, aircraft, or water craft for a localized or extremely broad decontamination depending upon the situation.

It is a still further object of the present invention to provide a system of the above type that uses a versatile compound that may safely be used to treat environments, equipment, personnel, animals, and food.

Toward the fulfillment of these and other objects and advantages, a system is disclosed in which a surface exposed to, suspected of being exposed to, or in imminent danger of being exposed to a biological warfare agent is treated with an effective amount of a quaternary ammonium compound to inactivate the biological warfare agent. The quaternary ammonium compound is preferably selected from the group consisting of an alkylpyridinium salt, a tetra-alkyl ammonium salt, and an allylalicyclic ammonium salt. The quaternary ammonium salt is more preferably an alkylpyridinium salt and is most preferably cetylpyridinium chloride (“CPC”). The compound is preferably present in solution in a weight percent that is substantially within a range of from approximately 0.5% to approximately 5.0% and is more preferably present in solution in a weight percent of approximately 1%. The compound may be applied as a mist, spray, or fog. Air or other gases that have been exposed to or that are suspected of being exposed to a biological warfare agent may also be treated by passing the air or gas through a filter impregnated with an effective amount of a quaternary ammonium compound to inactivate the biological warfare agent.

### BRIEF DESCRIPTION OF THE DRAWINGS

The above brief description, as well as further objects, features and advantages of the present invention will be more fully appreciated by reference to the following detailed description of the presently preferred but nonetheless illustrative embodiments in accordance with the present invention when taken in conjunction with the accompanying drawings, wherein:

FIG. 1 is a schematic representation of a system of the present invention;

FIG. 2 is a representation of a structural formula of CPC, the most preferred quaternary ammonium compound useful in practicing the present invention; and

FIG. 3 is a schematic representation of an alternate embodiment of a system of the present invention in which air is passed through a filter impregnated with a quaternary ammonium compound.

### DETAILED DESCRIPTION OF THE PREFERRED EMBODIMENT

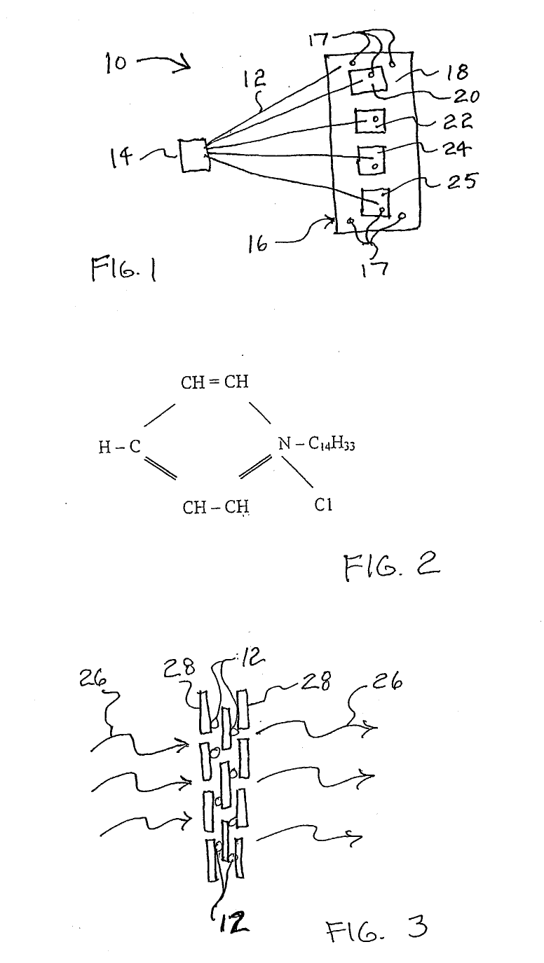

Referring to FIG. 1, the reference numeral 10 refers in general to a system of the above type in which a quaternary ammonium compound 12 is dispensed from a container 14 to treat surfaces 16 that have been exposed to a biological warfare agent 17. The surfaces 16 include but are not limited to environments 18, personnel 20, equipment 22, animals 24, and food 25.

The present system 10 involves the systematic decontamination of environments 18, personnel 20, equipment 22, animals 24, and food 25 using a quaternary ammonium compound 12 that inactivates a broad spectrum of biological warfare agents 17. The quaternary ammonium compound 12 is preferably selected from the group consisting of an alkylpyridinium salt, a tetra-alkyl ammonium salt, and an alkylalicyclic ammonium salt, is more preferably an alkylpyridinium salt, and is most preferably CPC. FIG. 2 shows a representation of a structural formula of cetylpyridinium chloride, C₂₁H₃₈NCl. The compound 12 is preferably present in solution in a weight percent that is substantially within a range of from approximately 0.5% to approximately 5.0% and is more preferably present in solution in a weight percent of approximately 1%. The solution preferably comprises water and the quaternary ammonium compound 12.

Table 1 provides a partial list of potential biological warfare agents 17 along with some estimates of infectious doses.

TABLE 1

| | |
|---|---|
| Agent | Infectious Dose |

Bacteria

| | |
|---|---|
| Bacillus anthracis—anthrax | 8,000-50,000 |
| Francisella tularensis—tularemia | 10-50 |
| Pseudomonas mallei—glanders | |
| Pseudomonas pseudomalli—meliodsis | |
| Brucella melitensis, suis, abortus—brucellosis | 10-100 |
| Yersinia pestis—plague | 100-500 |

Virus

| | |
|---|---|
| Flavivirus—Dengue fever | |
| Alphavirus—Equine encephalitis | 25 |
| Bunyavirus—Hataan, Crimean-Congo hemorrhagic fever | 1-10 |
| Filovirus—Ebola virus, Marburg virus | |
| Poxvirus—smallpox | 10-100 |

Rickettsia & Chiamidia

| | |
|---|---|
| Coxiella burnettii—Q-fever | 1-10 |
| Chlamydia psittaci-psittacosis | |
| Rickettsia rickettsil-Rocky Mountain spotted fever | |

Although the phrase “biological warfare agent” is used, it is understood that the present system is not limited to use during “war” and is instead useful in responding to attacks or contamination problems experienced during times of peace and war. For example, the present system is useful in responding to terrorist attacks using agents 17 such as the ones listed above regardless of whether the attacks are during times of peace or war and regardless of whether the targets are military or civilian.

The system may be used to treat any of a wide variety of surfaces 16. As used herein, the term “surface” 16 includes but is not limited to environments 18, personnel 20, equipment 22, animals 24, and food 25. The term “environment” includes but is not limited to terrain, structures, buildings, tents, streets, roads, runways, subways, and crops in the field. As used herein, the term “personnel” includes but is not limited to one or more military or civilian persons regardless of if or where they are employed and also includes but is not limited to clothing. As used herein, the term “equipment” includes but is not limited to machines, tools, gear, vehicles, aircraft, water craft, and storage containers.

In use, when a surface 16 has been exposed to a biological warfare agent 17, or when it is suspected that a surface 16 may have been or is in imminent danger of being exposed to a biological warfare agent 17, the surface 16 is treated with a quaternary ammonium compound 12. The surface 16 may be treated with the compound 12 in any number of ways. For example, the compound 12 may be atomized, fogged, misted, or sprayed from stationary or portable equipment, including but not limited to a vehicle, an aircraft, or a water craft 14. This allows for a localized or extremely broad application depending upon the situation. The quaternary ammonium compound 12 inactivates the agent 17 permanently and has a residual effect to prevent or reduce future contamination or recontamination. The compound 12 may be stored at or near possible military or civilian targets. Similarly, medical, rescue, and emergency response personnel may also maintain stores of the compound 12 for use in responding to attacks.

Referring to FIG. 3, in an alternate embodiment, the quaternary ammonium compound 12 may be incorporated into filtration systems for aircraft, tanks, trucks, automobiles, and other vehicles and shelters. Similarly, the quaternary ammonium compound may be incorporated into the filtration systems in protective masks. This may be accomplished in any number of ways. For example, air or other gases 26 that have been exposed to a biological warfare agent 17, may be passed through a filter 28 impregnated with the quaternary ammonium compound 12 to inactivate any such biological warfare agent 17 carried in the air 26. Similarly, water or other liquids may be filtered through a filtering media impregnated with the quaternary ammonium compound 12.

### EXAMPLE 1

An evaluation was performed of the effectiveness of 1% CPC as an antimicrobial agent against Bacillus subtilis, subsp. globigii, a surrogate of Bacillus anthracis. Due to the serious exposure hazards of working with Bacillus anthracis, Bacillus subtilis subsp. globigii is commonly researched as a non-pathogenic surrogate. Bacillus subtilis subsp. globigii was obtained from the American Type Culture Collection (ATCC 9372) and re-cultured and sporulated as per Sagripanti and Bonifacino (1996) (Comparative sporicidal effects of liquid chemical agents, Appl. Environ. Microbiol. 62(2): 545-51). A CPC solution (20%) was obtained from Safe Foods Corporation. A 2% working solution of this stock CPC solution was prepared by adding 100 mL of the 20% solution to 900 mL of sterile distilled water (final volume 1000 mL).

One mL of the Bacillus inoculum (ca. 10⁴ CFU/mL) was inoculated in 1 mL of the 2% CPC, providing an effective concentration of 1%. The organism was exposed to the CPC solution (1%) for 1, 15, and 30 minutes. Following this, the serial dilutions of the solution were plated onto appropriate agar media. The control sample consisted of inoculating 1 mL of Bacillus inoculum with 1 mL of sterile distilled water. Bacillus populations were enumerated before and after exposure to CPC or distilled water. The bacterial reductions in 1% CPC were compared with bacterial reductions in distilled water to determine the antimicrobial effectiveness of a 1% CPC solution after each of the three exposure times. The results of the evaluation are set forth in Table 2.

TABLE 2

| Treatment | Exposure | Time | Rep 1 | Rep 2 | Rep 3 | Total |
|---|---|---|---|---|---|---|
| Peptone | Before | 1 min. | 4.51 | 4.48 | 4.28 | 4.42 |
| Peptone | Before | 15 min. | 4.44 | 4.34 | 4.42 | 4.40 |
| Peptone | Before | 30 min. | 4.33 | 4.30 | 4.59 | 4.41 |
| Peptone | After | 1 min. | 3.67 | 3.63 | 3.67 | 3.66 |
| Peptone | After | 15 min. | 3.71 | 3.69 | 3.70 | 3.70 |
| Peptone | After | 30 min. | 3.75 | 3.70 | 3.69 | 3.71 |
| 2% CPC | Before | 1 min. | 4.32 | 4.21 | 5.38 | 4.64 |
| 2% CPC | Before | 15 min. | 4.33 | 4.36 | 4.40 | 4.36 |
| 2% CPC | Before | 30 min. | 4.30 | 5.02 | 4.40 | 4.57 |
| 2% CPC | After | 1 min. | 2.20 | 2.33 | 2.44 | 2.33 |
| 2% CPC | After | 15 min. | 2.30 | 2.63 | 2.60 | 2.51 |
| 2% CPC | After | 30 min. | 2.69 | 2.83 | 2.32 | 2.61 |

As reflected above, there was a Log₁₀ 2.31 reduction after 1 minute of exposure to 1% CPC, a Log₁₀ 1.85 reduction after 15 minutes of exposure to 1% CPC, and a Log₁₀ 1.96 reduction after 30 minutes of exposure to 1% CPC. By comparison, there was a Log₁₀ 0.76 reduction after 1 minute of exposure to 0.1% peptone, a Log₁₀ 0.70 reduction after 15 minutes of exposure to 0.1% peptone, and a Log₁₀ 0.70 reduction after 30 minutes of exposure to 0.1% peptone.

Other modifications, changes and substitutions are intended in the foregoing, and in some instances, some features of the invention will be employed without a corresponding use of other features. For example, the compound 12 may be applied only to personnel 20 who have been exposed to a biological warfare agent 17, rather than to all surfaces 16. Although treatment using atomizing, misting, spraying, or fogging is preferred, any number of ways may be used to bring the compound 12 into contact with the agent 17. The compound 12 may be combined with or used concurrently with other compounds, solutions, or the like for responding to contamination, suspected contamination, or imminent contamination by a biological warfare agent. A wide range of compositions and concentrations may be used. Of course, examples, measurements, and other numerical values are given by way of example only and are not intended to limit the scope of the invention. Accordingly, it is appropriate that the appended claims be construed broadly and in a manner consistent with the scope of the invention.

What is claimed is:

1. A method of reducing or preventing contamination of a surface by a biological warfare agent, comprising treating a surface contaminated by, suspected of being contaminated by, or in imminent danger of being contaminated by a biological warfare agent with an effective a amount of a quaternary ammonium compound to inactivate said biological warfare agent.

2. The method of claim 1 wherein said quaternary ammonium compound is selected from the group consisting of an alkylpyridinium salt, a tetra-alkyl ammonium salt, and an alkylalicyclic ammonium salt.

3. The method of claim 1 wherein said quaternary ammonium compound comprises an alkylpyridinium salt.

4. The method of claim 1 wherein said quaternary ammonium compound comprises cetylpyridinium chloride.

5. The method of claim 4 wherein said cetylpyridinium chloride is present in solution in a weight percent of approximately 1%.

6. The method of claim 1 wherein said biological warfare agent is selected from the group consisting of Bacillus anthracis; Francisella tularensis; Pseudomonas mallei; Pseudomonas pseudomalli; Brucella melitensis; suis, abortus; Yersinia pestis; Flavivirus; Alphavirus; Bunyavirus-Hataan; Filovirus; Poxvirus; Coxiella burnettii; Chlamydia psittaci; and Rickettsia rickettsil.

7. The method of claim 6 wherein said quaternary ammonium compound is selected from the group consisting of an alkylpyridinium salt, a tetra-alkyl ammonium salt, and an alkylalicyclic ammonium salt.

8. The method of claim 7 wherein quaternary ammonium salt is present in solution in a weight percent that is substantially within a range of from approximately 0.5% to approximately 5.0%.

9. The method of claim 6 wherein said quaternary ammonium compound comprises cetylpyridinium chloride.

10. The method of claim 9 wherein said cetylpyridinium chloride is present in solution in a weight percent of approximately 1%.

11. The method of claim 1 wherein said biological warfare agent comprises Bacillus anthracis.

12. The method of claim 11 wherein said quaternary ammonium compound is selected from the group consisting of an alkylpyridinium salt, a tetra-alkyl ammonium salt, and an alkylalicyclic ammonium salt.

13. The method of claim 11 wherein said quaternary ammonium compound comprises cetylpyridinium chloride.

14. The method of claim 13 wherein said cetylpyridinium chloride is present in solution in a weight percent that is substantially within a range of from approximately 0.5% to approximately 5.0%.

15. The method of claim 13 wherein cetylpyridinium chloride is present in solution in a weight percent of approximately 1%.

16. The method of claim 13 wherein said surface comprises personnel.

17. The method of claim 13 wherein said surface comprises equipment.

18. The method of claim 13 wherein said surface comprises an environment.

19. The method of claim 1 wherein said surface is treated by contacting said surface with a solution of said quaternary ammonium compound that has been atomized, misted, sprayed, or fogged.

20. The method of claim 13 wherein said surface is treated by contacting said surface with a solution of said cetylpyridinium chloride has been atomized, misted, sprayed, or fogged.

21. A method of reducing contamination of air by a biological warfare agent, comprising passing air contaminated by or suspected of being contaminated by a biological warfare agent through a filter impregnated with an effective amount of a quaternary ammonium compound to inactivate said biological warfare agent.

22. The method of claim 21 wherein said biological warfare agent is selected from the group consisting of Bacillus anthracis; Francisella tularensis; Pseudomonas mallei; Pseudomonas pseudomalli; Brucella melitensis; suis, abortus; Yersinia pestis; Flavivirus; Alphavirus; Bunyavirus-Hataan; Filovirus; Poxvirus; Coxiella burnettii; Chlamydia psittaci; and Rickettsia rickettsil.

23. The method of claim 21 wherein said biological warfare agent comprises Bacillus anthracis.

24. The method of claim 23 wherein said quaternary ammonium compound comprises cetylpyridinium chloride.

25. The method of claim 13 wherein said surface comprises water.

26. A method of reducing contamination of water by a biological warfare agent, comprising passing water contaminated by or suspected of being contaminated by a biological warfare agent through a filter impregnated with an effective amount of a quaternary ammonium compound to inactivate said biological warfare agent.

27. The method of claim 26 wherein said biological warfare agent is selected from the group consisting of Bacillus anthracis; Francisella tularensis; Pseudomonas mallei; Pseudomonas pseudomalli; Brucella melitensis; suis, abortus; Yersinia pestis; Flavivirus; Alphavirus; Bunyavirus-Hataan; Filovirus; Poxvirus; Coxiella burnettii; Chlamydia psittaci; and Rickettsia rickettsil.

28. The method of claim 26 wherein said biological warfare agent comprises Bacillus anthracis.

29. The method of claim 28 wherein said quaternary ammonium compound comprises cetylpyridinium chloride.

30. The method of claim 1 wherein said quaternary ammonium compound consists essentially of an alkylpyridinium salt.

31. The method of claim 1 wherein said quaternary ammonium compound consists essentially of cetylpyridinium chloride.

32. The method of claim 1 wherein said quaternary ammonium compound is present in solution, said solution consisting essentially of said quaternary ammonium compound and water.

33. The method of claim 30 wherein said alkylpyridinium salt is present in solution, said solution consisting essentially of said alkylpyridinium salt and water.

34. The method of claim 31 wherein said cetylpyridinium chloride is present in solution, said solution consisting essentially of said cetylpyridinium chloride and water.
